(19) United States
(12) Patent Application Publication
(10) Pub. No.: US 2002/0015468 A1
(43) Pub. Date: Feb. 7, 2002

(54) Computed tomography method involving conical irradiation of an object

(76) Inventors: Thomas Kohler, Norderstedt (DE); Michael Grass, Hamburg (DE)

Correspondence Address: U.S. Philips Corporation, 580 White Plains Road, Tarrytown, NY, 10591, US

(21) Appl. No.: 09/906,606

(22) Filed: Jul. 17, 2001

(30) Foreign Application Priority Data
Jul. 19, 2000 (DE) 10035138.7

(51) Int. Cl. A61B 6/03

(52) U.S. Cl. 378/4; 378/901

(57) ABSTRACT

The invention relates to a computed tomography method in which a series of CT data sets is continuously acquired along a closed, preferably circular trajectory while using a conical radiation beam. Because a CT data set acquired along such a trajectory is incomplete, artefacts occur; such artefacts are reduced in accordance with the invention by taking the following steps:

acquisition of at least one first, complete CT data set,

acquisition of a series of second CT data sets while the relative motion between the radiation beam and the object corresponds each time to a closed, preferably circular trajectory,

supplementing the data of the second data sets with data of the first data set, and

reconstruction of a series of CT images from the supplemented second CT data sets.

The invention relates to a computed tomography method wherein a conical radiation beam traverses an object and wherein CT data (CT=computed tomography) is acquired and CT images are reconstructed continuously. Such applications cannot employ helical trajectories (a trajectory is to be understood to mean herein the relative motion between the radiation beam and the object), because such trajectories imply a reversal of the direction of movement that would give rise to motional unsharpness in the reconstructed CT images.

Therefore, closed, preferably flat and notably circular trajectories are used for such applications. However, it is known that in the case of a circular trajectory complete data is acquired only for the slice which is situated in the plane of the trajectory. Therefore, such so-called “missing data” problems lead to the appearance of artefacts in the reconstructed images, said artefacts being more pronounced as the angle that is enclosed by the rays to the voxels to be reconstructed relative to the plane of the trajectory is larger.

It is an object of the present invention to provide a computed tomography method such that less pronounced artefacts occur. This object is achieved in accordance with the invention by means of a computed tomography method wherein a conical radiation beam traverses an object and which includes the following steps:

acquisition of at least one first, complete CT data set,

acquisition of a series of second CT data sets while the relative motion between the radiation beam and the object corresponds each time to a closed, preferably circular trajectory,

supplementing the data of the second data sets with data of the first data set,

reconstruction of a series of CT images from the supplemented second CT data sets.

Thus, in conformity with the invention first a first complete CT data set is acquired. This acquisition effectively takes place directly before the actual “fluoroscopic” CT examination. Subsequently, second CT data sets are continuously acquired along a second, preferably circular trajectory. As has already been explained, such data sets are incomplete and hence would give rise to the previously described artefacts in the reconstruction of CT images from such second data sets. Prior to the reconstruction, however, the data of the relevant second data set is supplemented by data of the first CT data set.

Because this first CT data set is complete by definition, the reconstruction from the supplemented data results in CT images that are free from said artefacts provided that no changes have occurred in the object region with which the second data set is associated (that is, no changes with respect to the instant at which the first data set has been acquired). If changes have occurred in this region, for example due to an incoming flow of contrast medium, the introduction of a surgical instrument or motions of the object, said artefacts also appear but in less pronounced form, because the regions of the object that have remained the same can no longer influence such artefacts.

It is to be noted that EP-OS 0860144 A2 already discloses a computed tomography method wherein first a first CT data set is acquired and subsequently a series of second CT data sets. Such a first data set is acquired along a helical trajectory; therefore, it is complete. EP-OS 0860144 A2 does not offer any further information as regards the trajectory along which the second data sets are acquired, so that it is not clear whether the second data sets are complete or incomplete.

However, CT images are reconstructed separately from the CT data sets. A CT image that is intended to represent the anatomy of the patient is reconstructed from the first data set and a CT image which is intended to show, for example, an instrument introduced into the patient is reconstructed from the second data set. The two CT images are combined so that the position of the surgical instrument within the patient can be recognized.

The second CT image, being reconstructed exclusively from the data of the second CT data set, therefore, contains to a full extent the artefacts that are due to the “missing data” problem. When, moreover, the second CT image is reconstructed in such a manner that the anatomical information that is in principle present therein is suppressed, changes in the anatomical region, for example due to a displacement of the patient, cannot be recognized in the combination image. This may have fatal consequences when the surgical instrument is intended to remove a tumor whose position has changed between the acquisition of the first and the second data set.

According to the invention, however, the artefacts are reduced and all changes (not only those that are due to, for example, the introduction of an instrument) in the imaged object region are effectively reproduced. The second data set can be supplemented with the data of the first data set in various ways before a CT image is reconstructed therefrom.

Claim 2 discloses a first, preferred solution. Therein, first the first CT image is reconstructed. After that the so-called “object function” is known, that is, the spatial distribution of the attenuation of the radiation within the object. Therefore, virtual CT data for the trajectory on which the acquisition of the second data set is based can be derived from the first CT image. If no changes occur in the object region covered by the second data set, such virtual data must be identical to the CT data of the second CT data set. The reconstruction of a CT image from the difference between the virtual CT data applicable to the same rays and the CT data of the second data set, therefore, yields a difference CT image which reproduces merely the changes in the object region.

Such a CT image makes the orientation difficult in many cases. However, when in conformity with claim 3 the first CT image is superposed on the difference CT image with a suitable weight, orientation becomes readily possible in the superposed image.

Claim 4 discloses a second possibility of supplementing the CT data of the second CT data set with data of the first CT data set prior to the reconstruction. It is known that the CT data acquired along a trajectory can be described in the so-called Radon space. In the case of a circular trajectory, the Radon values that can be calculated fill a ring-shaped torus in the Radon space. For complete reconstruction, however, the values should be known in an ellipsoid of revolution or in a spherical region of the Radon space. The region of the Radon space that is necessary for complete reconstruction is obtained when the Radon values that cannot be calculated on the basis of the second data set and are necessary for a complete reconstruction are supplemented with Radon values of the first data set before the reconstruction of a CT image from these Radon values.

Claim 5 discloses a preferred possibility for the acquisition of the first data set. Granted, a complete data set can also be acquired other trajectories (for example, along two intersecting circular trajectories), it is particularly simple to realize a helical relative motion and reconstruction methods that are suitable in this respect are also known.

Claim 6 defines a computed tomography apparatus which is suitable for carrying out the method claimed in claim 1, and claim 7 relates to a computer program that is capable of controlling the control unit of a computed tomography apparatus in such a manner that the computed tomography method in accordance with the invention is executed.

The invention will be described in detail hereinafter with reference to the drawings. Therein:

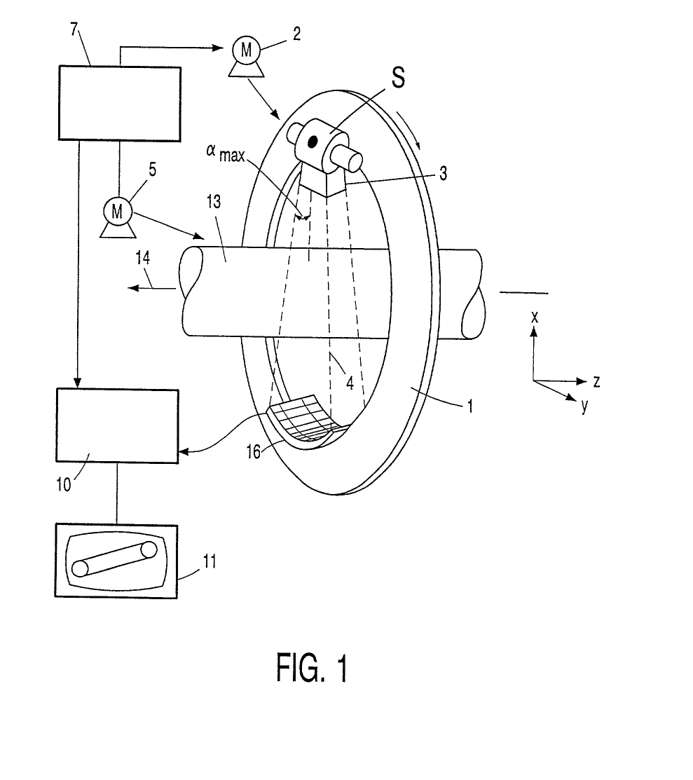

FIG. 1 shows a computed tomography apparatus which is suitable for carrying out the invention,

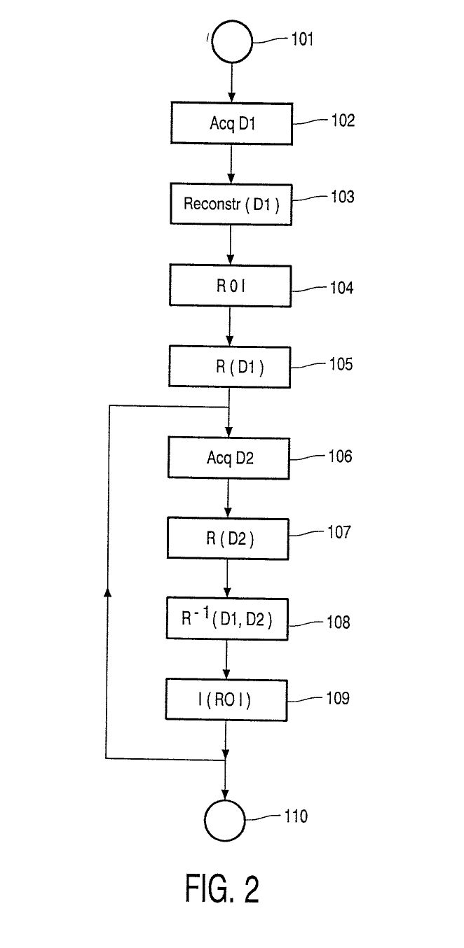

FIG. 2 shows a flow chart illustrating a first version of the invention,

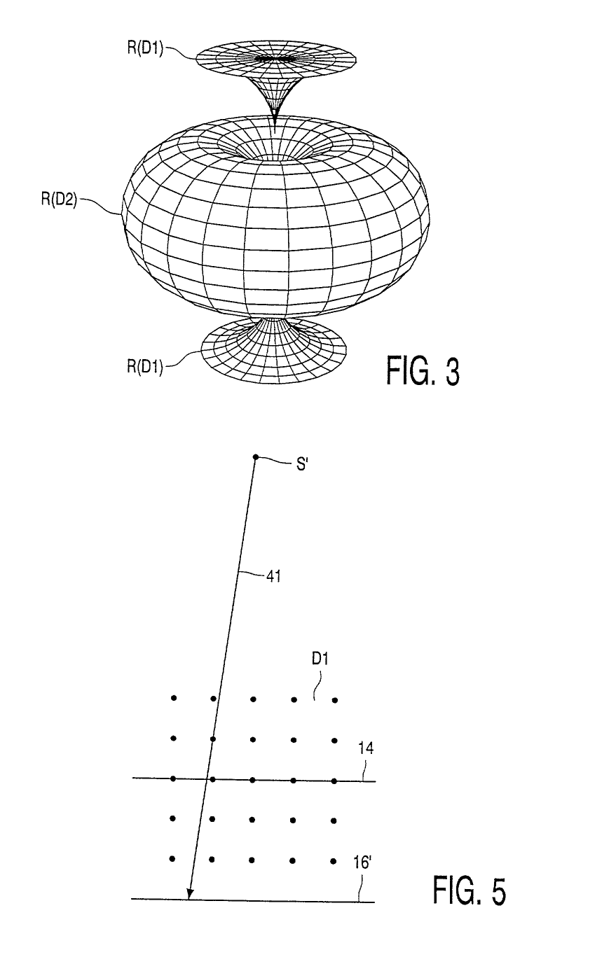

FIG. 3 shows the supplementary Radon values in the Radon space,

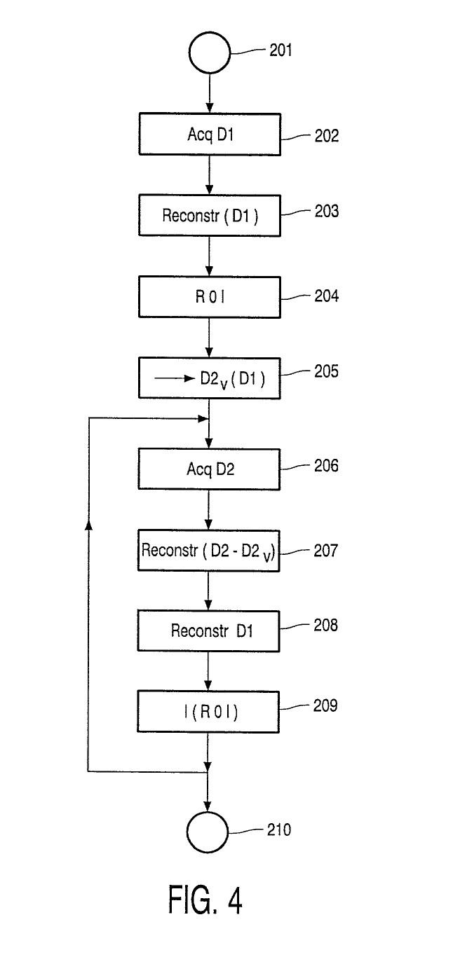

FIG. 4 shows a flow chart illustrating a second version of the method in accordance with the invention, and

FIG. 5 illustrates the calculation of the virtual CT data.

The computed tomography apparatus which is shown in FIG. 1 includes a gantry 1 which is capable of rotation about an axis of rotation 14 which extends parallel to the z direction of the co-ordinate system shown in FIG. 1. To this end, the gantry is driven at a preferably constant but adjustable angular speed by a motor 2. A radiation source S, for example an X-ray source is mounted on the gantry. The X-ray source is provided with a collimator arrangement 3 which forms a conical radiation beam 4 from the radiation produced by the radiation source S, that is, a radiation beam which has a finite dimension other than zero in the z direction as well as in a direction perpendicular thereto (that is, in a plane perpendicular to the axis of rotation).

The radiation beam 4 enters an examination zone 13 in which an object may be present, for example a patient on a patient table (both of which are not shown). The examination zone 13 is shaped as a cylinder. After having traversed the examination zone 13, the X-ray beam 4 is incident on a two-dimensional detector unit 16 which is mounted on the gantry 1 and includes a plurality of adjacently arranged detector rows, each of which includes a plurality of detector elements. The detector rows are situated in planes extending perpendicularly to the axis of rotation 14, that is, preferably on an arc of a circle around the radiation source S. However, they may also have a different shape; for example, they may describe an arc of a circle about the axis of rotation 14 or be straight. In each position of the radiation source each detector element that is struck by the radiation beam 4 produces a measuring value for a ray of the radiation beam 4. The measured values are also referred to as CT data hereinafter.

The angle of aperture of the radiation beam 4, being denoted by the reference αₘₐₓ (the angle of aperture is defined as the angle enclosed by a ray which is situated at the edge of the radiation beam 4 in a plane perpendicular to the axis of rotation 14 relative to a plane defined by the radiation source S and the axis of rotation 14), determines the diameter of the cylindrical examination zone 13 within which the object to be examined must be present during the acquisition of the CT data.

The examination zone 13, or the object or the patient table, can be displaced, by means of a motor 5, that is, parallel to the axis of rotation 14 or parallel to the z axis. Equivalently, however, the gantry could also be displaced in this direction; only the relative shift is essential. When the motors 5 and 2 run simultaneously, the radiation source S and the detector unit 16 describe a helical trajectory relative to the examination zone 13. However, when the motor 5 for the displacement in the z direction is inactive and the motor 2 rotates the gantry 1, a circular trajectory is obtained for the radiation source S and the detector unit 16 relative to the examination zone 13.

The measuring values or CT data acquired by the detector unit 16 are applied to an image processing computer 10 which reconstructs the absorption distribution in a part of the examination zone 13 therefrom, for example for display on a monitor 11. The two motors 2 and 5, the image processing computer 10, the radiation source S and the transfer of the CT data from the detector unit 16 to the image processing computer 10 are controlled by a control unit 7.

FIG. 2 illustrates the execution of a CT method that can be carried out by means of the computed tomography apparatus shown in FIG. 1. After the initialization in the block 101, the motors 2 and 5 are simultaneously switched on, so that the radiation source S, or the detector unit 16, follows a helical path relative to the examination zone or the object present therein. During this relative motion the radiation source S is switched on and the measuring values then acquired are stored in a memory of the image processing computer 10.

Subsequently, in the step 103 a first CT image is reconstructed from the first CT data set thus acquired. A suitable reconstruction method is known, for example from U.S. application Ser. No. 09/368,850 (PHD 98-086).

During the next step 104 the region of the object that is of interest to the examination (ROI=region of interest) and is to be continuously imaged during the further examination is defined. The gantry is then shifted parallel to the axis of rotation until the region of interest can be covered by the radiation beam 4 so as to be reconstructed while using a circular trajectory (that is, while the radiation source and the object rotate relative to one another about the axis of rotation, but are not displaced in the direction of the axis of rotation).

During the step 105 the Radon values that will be missing in the acquisition of a second data set along the predetermined trajectory, because they are situated outside the ring-shaped torus in the Radon space in which Radon values occur for the second data set, are calculated from the first CT data set.

This is illustrated in FIG. 3 which shows the ring-shaped torus R(D2) which encloses the points in the Radon space for which Radon values can be calculated from the second CT data set. In this respect it is assumed that the axis of rotation extends vertically in FIG. 3. Above and below the torus R(D2), that is, on the symmetry axis thereof, there are situated the points R(D1) in the Radon space which supplement the ring-shaped torus so that it forms a sphere (for this purpose the faces that are remote from the torus should be dome-shaped and not flat as assumed for the sake of simplicity in FIG. 3). In the step 105 the Radon values are calculated for the points in the regions R(D1) of the Radon space. As is known, a Radon value for a point in the Radon space is calculated as the surface integral of the attenuation of the radiation in a plane which contains said point and extends perpendicularly to the connecting line between this point and the zero point of the Radon space.

In the step 106 only the motor 2 is switched on, so that the radiation source and the detector unit 16 rotate about the axis of rotation 14 and hence about the examination zone 13 or the object present therein. The radiation of the radiation source S is then switched on and the CT data acquired by the detector elements of the detector unit 16 is stored. The totality of data acquired during one revolution of the radiation source constitutes a second CT dataset.

In the step 107 the Radon values for all points within the annular torus R(D2) (see FIG. 3) are calculated from said second data set. After all Radon values required for complete reconstruction have been calculated for the points in the Radon space, an inverse Radon transformation is performed in the step 108 over said Radon values derived from the second data set and partly from the first data set. Such inverse Radon transformation is known, for example from the book by F. Natterer “The mathematics of computerized tomography” (ISBN 3 519 02103 X), chapter II.2).

This reconstruction step yields a (three-dimensional) CT image which represents the object function, or the attenuation of the radiation in the examination zone, as a function of the location. This CT image is suitably displayed on the monitor 11 in the step 109, for example in the form of a slice image for a slice contained in the three-dimensional region.

Subsequently, the loop consisting of the steps 106 to 109 is completed again so as to reconstruct a further CT image from the data then acquired (supplemented with the Radon values R(D1)). The acquisition of the data could commence already before the reconstruction and/or display of the CT image in the steps 108 and 109 is finished. When the examination has been completed, the method terminates in the step 110.

A further, preferred version of the invention will be described in detail hereinafter with reference to the FIGS. 4 and 5. The steps 201 to 204 therein correspond to the steps 101 to 104 of the method described with reference to the FIGS. 2 and 3. In the step 205 virtual CT data is calculated from the first CT image reconstructed in the step 203 for a circular trajectory (used during the subsequent acquisition of the second data set).

This step is illustrated in FIG. 5 in which a respective point symbolizes the voxels for which the object function or the attenuation of the radiation has been determined in the form of the first CT image. These points define a regular, preferably Cartesian, three-dimensional grid (further voxels are situated above and below the plane of drawing of FIG. 5). The Figure also shows one of the positions S′ occupied by the radiation source S during the acquisition of further data sets along a circular trajectory relative to the part of the examination zone to be reconstructed. Finally, the associated position 16′ of the detector unit 16 is also shown.

When the position S′ is linked to a detector element of the detector unit in the position 16′ by way of a straight line or a ray 41, the attenuation of the radiation along the ray 41 can be calculated from the attenuation values determined for the various voxels in the first data set. When this calculation is performed for all detector elements of the detector unit 16 and for each position of the radiation source on the circular trajectory, a set of virtual CT data is obtained which corresponds to the second CT data set acquired along a circular trajectory in the subsequent step 206 (corresponding to the step 106 of the method described with reference to the FIGS. 2 and 3).

When no changes have occurred in the region imaged by the second data set between the acquisition of the first data set and the second data set (and if the reconstruction of the first CT image from the first data set and the calculation of the virtual CT data from the first CT image is very exact), the associated CT data of the second data set will correspond to the virtual data for all rays. However, when a change has occurred, for example due to the introduction of a surgical instrument (for example, a biopsy needle) into the region being imaged or because contrast medium flows into this region or a shift of the object has occurred at least locally between the acquisition of the two data sets, the virtual CT data will deviate from the CT data of the second data set.

The step 207 utilizes such differences between the CT data of the second data set and the virtual CT data (each time for the same ray) for the reconstruction of a difference CT image which represents the changes in the region covered by the second CT data set with respect to the instant of acquisition of the first CT data set. Reconstruction is performed by means of so-called filtered backprojection. A reconstruction method that is suitable in this respect is known from U.S. application Ser. No. 09/400,763 (PHD 98-111).

Generally speaking, anatomical orientation in such a difference CT image is comparatively difficult; therefore, in a further reconstruction step 208 the first CT image reconstructed from the first CT data set is superposed on the difference CT image with a suitable weighting factor. The superposed image is displayed in the step 209. Subsequently, the steps 206 to 209 are repeated as often as necessary, after which the execution of the method is stopped (step 210).

1. A computed tomography method wherein a conical radiation beam traverses an object and which includes the following steps:
acquisition of at least one first, complete CT data set,
acquisition of a series of second CT data sets while the relative motion between the radiation beam and the object corresponds each time to a closed, preferably circular trajectory,
supplementing the data of the second data sets with data of the first data set,
reconstruction of a series of CT images from the supplemented second CT data sets.

2. A computed tomography method as claimed in claim 1, characterized in that it includes the following steps:
reconstruction of a first CT image from the first CT data set,
calculation of virtual CT data from the first CT image for the radiation traversing the object during the acquisition of one of the second data sets,
calculation of the differences between the virtual CT data and the CT data of the second CT data set acquired for the same rays,
reconstruction of a difference CT image from the differences.

3. A computed tomography method as claimed in claim 2, characterized in that the first CT image and the difference CT image are superposed.

4. A computed tomography method as claimed in claim 1, characterized in that it includes the following steps:
calculation, from the first CT data set, of the Radon values that cannot be calculated from the second data set,
calculation of the Radon values that can be calculated from the relevant second data set,
inverse Radon transformation of the Radon values determined from the two data sets.

5. A computed tomography method as claimed in claim 1, characterized in that the radiation beam and the object perform a relative motion along a helical path during the acquisition of the first CT data set.

6. A computed tomography apparatus for carrying out the method claimed in claim 1, including
a radiation source for generating a conical radiation beam,
a detector unit which is coupled thereto for the acquisition of CT data,
a drive device for making an object present in the examination zone and the radiation source perform a rotation relative to one another about an axis of rotation and/or for making these elements move parallel to the axis of rotation,
a reconstruction unit for reconstructing the spatial distribution of the absorption within the examination zone from the measuring data acquired by the detector unit, and
a control unit for controlling the radiation source, the detector unit, the drive device and the reconstruction unit,
characterized in that the control unit is programmed in such a manner that the following sequence is executed:
acquisition of at least one first, complete CT data set,
acquisition of a series of second CT data sets while the relative motion between the radiation beam and the object corresponds each time to a closed, preferably circular trajectory,
supplementing the data of the second data sets with data of the first data set,
reconstruction of a series of CT images from the supplemented second CT data sets.

7. A computer program for a control unit (7) for controlling a radiation source (S), a detector unit (16), a drive device and a reconstruction unit (10) of a computed tomography apparatus so as to carry out the method claimed in claim 1 as follows:
generating, using a radiation source (S), a conical radiation beam (4) which traverses an examination zone (13) or an object present therein,
acquisition of at least one first, complete CT data set,
acquisition of a series of second CT data sets while the relative motion between the radiation beam and the object always corresponds each time to a closed, preferably circular trajectory,
supplementing the data of the second data sets with data of the first data set, and
reconstruction of a series of CT images from the supplemented second CT data sets.
